(19) United States
(12) Patent Application Publication
(10) Pub. No.: US 2004/0001809 A1
(43) Pub. Date: Jan. 1, 2004

(54) Methods and apparatus for enhancing a response to nucleic acid vaccines

(75) Inventors: Axel F. Brisken, Fremont, CA (US); Robert Zuk, Atherton, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: PHARMASONICS, INC., Sunnyvale, CA

(21) Appl. No.: 10/184,143

(22) Filed: Jun. 26, 2002

(51) Int. Cl. A61K 48/00; A61N 1/30

(52) U.S. Cl. 424/93.21; 514/44; 604/20

(57) ABSTRACT

The immune response achieved by the administration of nucleic acid vaccines is enhanced by the application of vibrational energy to the inoculated tissue region. The vibrational energy is selected to enhance transfection of the tissue without substantial tissue damage, relying on a mechanical effect of the vibrational energy. Additionally, vibrational energy intended to mechanically injure the tissue via a thermal effect may also be applied.

### BACKGROUND OF THE INVENTION

1. Field of the Invention

The present invention relates generally to medical apparatus and methods. More particularly, the present invention relates to the use of vibrational energy for enhancing an immune response elicited by DNA and RNA vaccines.

Conventional vaccines, typically comprising killed or attenuated bacterial or viral pathogens, have been successfully developed against numerous infectious diseases, such as smallpox, typhus, tetanus, measles, hepatitis A and B, and other dangerous infections. Such vaccines have not been successfully developed yet for numerous other conditions, such as malaria, HIV infection, herpes, hepatitis C, and others. Thus, much research and development continues on the development of improved vaccines.

One promising approach that has been under development for the past decade involves the administration of DNA or RNA encoding an immunogen of the pathogen to a susceptible host. The genetic material, typically in the form of a plasmid, is injected into solid tissue where it is taken up by cells, transfecting the cells to produce the immunogen. In turn, the immunogen initiates a protective humoral and cellular response. Injection is commonly achieved using a needle, gene gun, or the like. Although some success has been achieved, the ability to successfully achieve immunity with a wide variety of DNA and RNA vaccines has remained elusive.

For these reasons, it would be desirable to provide improved methods and apparatus for administering DNA and RNA vaccines to humans and other animal hosts. Such methods and apparatus should desirably achieve successful transfection of cells in the human or animal host as well as relatively high levels of expression of the immunogen(s) encoded by the vaccines. Moreover, the methods and apparatus should be able to induce an enhanced immune response in the patient, preferably with elevated humoral (antibody) and/or cellular responses compared to other delivery techniques. Additionally, the methods and apparatus should be relatively convenient to use, relatively painless to the patient, and economically attractive to the healthcare provider. At least some of these objectives will be met by the invention described hereinafter.

2. Description of the Background Art

WO 01/23537 A1 and WO 00/45823 A1 both describe the administration of DNA vaccines enhanced by electroporation. Co-pending application Ser. No. 09/435,095, describes widebeam ultrasonic transducer apparatus which are useful for enhancing transfection of injected nucleic acid constructs. The devices described in this co-pending application will be useful for performing at least some of the methods of the present application, optionally in combination with novel ultrasonic transducer assemblies.

### BRIEF SUMMARY OF THE INVENTION

The present invention provides for an enhanced immune response in animal hosts, particularly humans, inoculated with nucleic acid (DNA or RNA) vaccines. In particular, the present invention relies on applying vibrational energy to a target site within solid tissue where the animal has been inoculated with the vaccine. In a first aspect of the present invention, it has been found that the application of vibrational energy under first conditions selected to enhance transfection of cells to produce antigen without significant tissue damage results in an enhancement of at least the humoral immune response in the animal compared to the response when the animal is inoculated without the application of vibrational energy. Increases in the humoral response from 3-fold to 5-fold have thus far been demonstrated.

In a second aspect of the present invention, it has been found that the humoral and possibly the cellular immune response may be further enhanced by additionally applying vibrational energy under conditions selected to produce tissue damage, particularly thermal tissue damage. It is believed that the additional application of vibrational energy acts primarily to cause an inflammatory response to the injury which has a synergistic effect with the primary response to the antigen production. While this is believed to be the mechanism by which the present invention achieves its desired effect, applicants do not wish to be bound by this speculation and claim the methods and apparatus set forth herein, regardless of the mechanisms by which they work.

While it will be preferred to utilize vibrational energy applied under both the first conditions and the second conditions, such conditions might also be combined with other nucleic acid vaccine delivery modes to achieve improved results. For example, a nucleic acid vaccine may be delivered to an animal host who is exposed to vibrational energy under the first conditions. The host may then be subjected to other energy-mediated conditions in order to cause an injury response which will further enhance the immune response of the patient. Alternatively, the nucleic acid vaccine may be administered to the host through a conventional inoculation protocol, such as injection, ballistic delivery, or the like, followed by the application of vibrational energy under the second conditions to cause thermal damage to the tissue in the region of the inoculation. Thus, the description and claims herein are intended to cover situations where the application of vibrational energy under either the first conditions or the second conditions is combined with other routes for inoculating the animal host with the nucleic acid vaccine.

Thus, a method according to the present invention for enhancing an immune response in an animal host, such as a human, comprises introducing nucleic acids encoding one or more immunogens to a target site in tissue. Vibrational energy is then applied to the target site in order to enhance the response. The phrases “nucleic acids encoding one or more immunogens” and “nucleic acid vaccine” are meant to-cover therapeutic and prophylactic compositions commonly referred to as DNA and/or RNA vaccines. The nucleic acid vaccines will typically encode a protein or peptide of a pathogen, where the protein or peptide is capable of inducing an immune response, usually including both a humoral immune response and a cellular immune response. The pathogen may be any type of human or animal pathogen, typically consisting of a bacterium, a fungus, a yeast, a protozoan, a virus, or the like. Exemplary bacteria and other pathogens include Clostridium, Vibrio, Nocardia, Corynebacterium, Listeria, Legionella, Bacilli, Staphylococcus; Streptococci, Borrelia, Mycobacterium, Neisserium and Trepanema bacterium. Exemplary fungus include Dermatophyte, Pneumocystis, Trypanosoma, Plasmodium, Candida, Cryptococcus, Histoplasma, Coccidioide, an Amoeba and Schistosome. Exemplary viral pathogens include parvovirus, an orthomyxovirus, paramyxovirus, picornavirus, papovirus, herpesvirus, togavirus, and retrovirus, with human immunodeficiency virus (HIV) being an exemplary retrovirus or RNA virus.

In all cases, the nucleic acid vaccine may encode more than one protein or peptide from the target pathogen, or from more than one target pathogen. For example, for HIV vaccines, the vaccine may encode the gag protein or various peptides or fragments thereof. The HIV vaccines may further encode the env protein or peptides or fragments thereof. The nucleic acid vaccines may comprise DNA which has been codon-optimized for the gag-encoding region and/or the env-encoding regions. The vaccines for other RNA viruses may similarly be embodied as DNA structures with codon-optimized sequences. Exemplary immunogens will be administered in plasmid form and may be incorporated together with conventional adjuvants, proteins, lipids, or the like. The nucleic acid vaccines may be introduced by conventional techniques including intramuscular injection, intradermal injection, ballistic delivery, and the like. In certain circumstances, the nucleic acid vaccine could also be delivered by other enhanced techniques, such as electroporation, generally as described above in the cited PCT publications, the full disclosures of which are incorporated herein by reference.

The vibrational energy applied to a target site in the tissue usually comprises ultrasonic energy which is directed from an ultrasonic transducer having characteristics and driven with excitation energy selected to enhance the immune response. In particular, a first transducer will be selected to operate at first conditions selected to provide a mechanical effect in the cells which enhances transfection of the cells to produce energy without significant tissue damage. Exemplary vibrational conditions are set forth in Table 1.

TABLE 1
First (Mechanical) Conditions

| | Freq. | Intensity | Duty Cycle | | |
|---|---|---|---|---|---|
| | (MHz) | (SPPA) | (%) | MI | TI |
| General | 0.02-5 | 0.5-10⁴ | 0.1-50 | 0.5-20 | <8 |
| Preferred | 0.1-2 | 50-300 | 0.5-10 | 1.5-3.5 | <4 |

Additionally or alternatively, the ultrasonic or other vibrational transducer will be driven at second conditions selected to produce tissue damage, usually thermal tissue damage, to stimulate the immune response to the antigen produced by the transfected cells. Exemplary second (thermal) conditions are set forth in Table 2.

TABLE 2
Second (Thermal) Conditions

| | Freq. | Intensity | Duty Cycle | | |
|---|---|---|---|---|---|
| | (MHz) | (SPPA) | (%) | MI | TI |
| General | 1-15 | 10-2000 | 10-100 | 0.1-2 | 10-5000 |
| Preferred | 2-8 | 50-500 | 100 | 0.2-1 | 20-1000 |

The mechanical index (MI) is a measure of the peak amplitude or pressure of the acoustic wave form, and as such, is independent of the burst duration and time during which the ultrasound transducer is turned on. The thermal index (TI) is a measure of the rate of power delivery to the target tissue and of the duration of exposure. The values of MI and TI in Tables 1 and 2 above represent asymptotic values, since the circulation of blood through tissue cools the tissue and the temperature will increase over time. The particular value of TI employed in the methods of the present invention may be low or high, depending principally on the duration of exposure which in turn depends on the degree of thermal injury required to achieve a desired level of transfection.

In preferred aspects of the present invention, the target site to which the nucleic acid vaccine has been introduced will be exposed to vibrational energy under both the first conditions and the second conditions as set forth in Tables 1 and 2. The volume of nucleic acid vaccine introduced to the target site may vary widely, typically being in the range from 0.05 mL to 5 mL, usually in the range from 0.1 mL to 1 mL, and often in the range from 0.2 mL to 0.4 mL. When delivered by injection, the DNA vaccine will typically be in a liquid form, optionally with an adjuvant present. The vaccine will permeate target region typically having a volume in the range from 0.1 cm³ to 5 cm³, usually from 0.2 cm³ to 2 cm³, and often from 0.5 cm³ to 1 cm³.

Vibrational energy according to the present invention may be exposed to the target site and tissue which receives the nucleic acid vaccine either before, after, or simultaneously with introduction of the nucleic acid vaccine. Preferably, the exposure to vibrational energy will occur simultaneously and/or within 2 minutes after the introduction of the vaccine. In some cases, however, it may be possible to inject or otherwise introduce the nucleic acid vaccine before the application of vibrational energy or well after the two minute window defined above. The volume of tissue to which the vibrational energy is delivered may be the same as, larger than, or in some instances smaller than the volume to which the nucleic acid vaccine has been introduced. The volumes of the first and second (as well as optionally subsequent) applications of vibrational energy may also differ, as discussed below. In general, the vibrational energy may be applied to target tissue volumes in the range from 0.1 cm³ to 5 cm³, usually, from 0.2 cm³ to 2 cm³, and typically from 0.5 cm³ to 1 cm³.

The vibrational energy will be applied to a volume of tissue within or surrounding the target site which receives the nucleic acid vaccine. For example, in some instances it will be desirable to expose the target site to vibrational energy under the first conditions, where the vibrational energy is applied to a volume which is at least as large as the volume which receives the vaccine. Vibrational energy under the second conditions may then be more narrowly focused within the target site, e.g., it may have a much smaller applied volume than the volume of tissue which received the vibrational energy under the first conditions. The present invention is not limited to such delivery, however, and there may be other instances where it is desirable to apply vibrational energy under the second conditions over a greater volume than receives vibrational energy under the first conditions. The delivery of vibrational energy under the first and second conditions may occur simultaneously, or in any other order, so long as vibrational energy under the first conditions is delivered so that it can enhance transfection of the tissue cells and vibrational energy under the second condition is delivered so that it can initiate an injury response which enhances the immune response mediated by expression of the immunogens encoded by the nucleic acid vaccine.

In certain specific embodiments, vibrational energy under the first conditions may be delivered to a target site having a volume in the range from 0.1 cm³ to 5 cm³, a thermal index selected (based on the period of time of delivery) to raise the tissue temperature by a value in the range from 0° C. to 4° C., and a mechanical index in the range from 0.5 to 20. The delivery of vibrational energy under the second conditions may then comprise a target site having a volume in the range from 0.01 cm³ to 1 cm³, a thermal index which causes a temperature rise in the range from 10° C. to 40° C., and a mechanical index in the range from 0.1 to 2. Usually, delivery of vibrational energy under at least the first conditions (and usually both the first and second conditions) will comprise delivering ultrasonic energy having a field intensity which varies by less than 6 db across the width and depth of the field and across the depth of the target site.

Systems according to the present invention for enhancing an immune response in an animal comprise a means for administering a nucleic acid vaccine to a target site in the animal, such as a needle, ballistic delivery device, needles injector (gas-propelled), or the like. The system will further comprise means for applying vibrational energy to the target site and a source of nucleic acid vaccine coupleable to the administering means, e.g., a needle and syringe filled with the nucleic acid vaccine. The vibrational energy means may comprise a single transducer for applying both the first conditions and the second conditions, but will more often comprise a first transducer which directs vibrational energy at the target site under the first conditions (which enhance transfection of the cells in the target site) and a second transducer which directs vibrational energy at the target site under the second conditions (which produce tissue damage to stimulate an immune response).

The first and second transducers may be mounted together within a single housing, usually a hand-held housing which permits the user to manually engage the transducers externally against a skin surface overlying the tissue to which the nucleic acid vaccine has been administered. Alternatively, the first and second transducers may be separately housed so that they may be used separately, either sequentially or simultaneously, by individually engaging them externally against the patient's skin. Optionally, the needle, ballistic delivery, or other nucleic acid administering means may be combined in the single housing which incorporates both the first and second transducers, or either of the individual housings incorporating the transducers when they are mounted separately.

The first transducer and second transducer will operate as described above, typically at the conditions set forth in Tables 1 and 2 respectively. When incorporated in a single housing, the transducers may be arranged in any convenient manner. For example, the second transducer may be arranged annularly about the first transducer, where the second transducer focuses thermally disruptive vibrational energy within a region which lies inside of a beam produced by the first transducer. A variety of other specific apparatus designs will also be available.

The DNA vaccine source may comprise any of the immunogens set forth above with respect to the methods of the present invention.

Although the methods and systems of the present invention preferably rely on delivering energy to the target tissues under two distinct sets of conditions which are optimized to enhance transfection on the one hand, and to induce an inflammatory response on the other, in some circumstances it may be possible to deliver energy from a single transducer and/or under a single set of conditions in order to enhance transfection and simultaneously induce an inflammatory response. The single set of conditions will be selected as a compromise, but in some instances may be sufficiently effective to provide for the benefits described herein.

### BRIEF DESCRIPTION OF THE DRAWINGS

FIGS. 1A-1C illustrate a method according to the principles of the present invention where a nucleic acid vaccine is injected into a target site in solid tissue, followed by the separate application of vibrational energy under the first conditions and vibrational energy under the second conditions.

FIG. 2 illustrates an exemplary design for a “thermal” transducer comprising a spherically concave transducer.

FIGS. 3A and 3B illustrate the different focal lengths and focused beam widths that can be achieved using the spherical transducers of FIG. 2.

FIG. 4 illustrates the first embodiment of a combination device comprising both a “mechanical” transducer and a “thermal” transducer constructed in accordance with the principles of the present invention.

FIG. 5 illustrates a second exemplary design for a combination device constructed in accordance with the principles of the present invention.

FIG. 6 illustrates a third exemplary design for of a combination device constructed in accordance with the principles of the present invention.

FIG. 7 illustrates a composite annular array device, shown in section, where the individual elements of the array may be separately powered in order to deliver both mechanical and thermal vibrational energy according to the methods of the present invention.

FIG. 8 illustrates control circuitry suitable for use with the array transducer of FIG. 7.

### DETAILED DESCRIPTION OF THE INVENTION

The following terms and phrases will have the definitions set forth when used in the claims and specification herein.

The terms “animal” and “animal host” will include all human and other vertebrate animals which are capable of eliciting an immune response when challenged with an immunogen. While the present invention will find its greatest use with humans, it will also be useful for protecting livestock, pets, and other domesticated or wild animals.

The phrase “immune response” refers collectively to the humoral and cellular responses of the human or other animal host when challenged with an immunogen such as those encoded by the nucleic acid vaccines of the present invention. The enhanced immune response of the present invention refers to an increase in either or both of the humoral and cellular (usually at least the humoral) immune responses.

The phrase “nucleic acid vaccine” and “nucleic acids” includes both DNA and RNA constructs of the type which encode an immunogen, or a fragment or a peptide thereof, and which may be administered to the human or other animal host. Conveniently, the DNA or RNA may be part of a self-replicating plasmid or other vector which permits production of the nucleic acid construct in an in vitro system. For RNA viruses, the nucleic acid vaccines may comprise DNA which has been transposed from the RNA coding sequence so that it may be replicated and prepared in a DNA form. The immunogens encoded by the nucleic acid vaccines may form proteins, peptides, protein fragments, or the like, from virtually any infectious pathogen, including at least those pathogens listed hereinabove.

The term “transfection” refers to the uptake of the nucleic acids from the nucleic acid vaccine by the cells of the human or other animal host in the region where the vaccine has been introduced, by injection or by other means. In particular, cells transfected by the vaccine will produce the immunogen, preferably as a cell surface protein and/or by releasing the protein into tissue and vascular circulation. By producing the immunogen as a cell surface antigen, a cellular response may be achieved. By releasing the immunogen into vascular circulation, a humoral response may be achieved.

The phrase “target site” refers to the region or volume within the solid tissue of the human or other animal host that is being inoculated with a nucleic acid vaccine. Preferred target sites will be in the legs, arms, other large muscle sites, solid organs, and the like. The volume of the target site will depend on the volume of nucleic acid vaccine delivered, including any carrier, adjuvant, or other dispersion means. Typically, the volume of the target site will be in the range from 0.1 cm³ to 5 cm³, usually from 0.2 cm³ to 2 cm³.

The phrase “vibrational energy” refers to ultrasonic, acoustic, and other mechanical forms of vibration which may be applied to the target site within the tissue of the human or other animal host being treated by the methods herein. Usually, the vibrational energy will be ultrasonic energy delivered under the conditions set forth in Tables 1 and 2, hereinabove.

The phrase “mechanical effect” refers to the biological effect of the ultrasonic energy delivered to the target tissue, typically within the conditions set forth in Table 1 hereinabove. Ultrasonic energy within these parameters will be characterized by cavitational, pressure, and high frequency characteristics with minimal thermal contribution, i.e., heat due to absorption of energy or energy conversion to heat. For achieving transfection with the nucleic acid vaccines of the present invention, it is desirable to use ultrasonic energy with mechanical biological effects rather than thermal biological effects. Such energy will produce a temperature rise below 4° C., i.e. from 0° C. to 4° C.

The phrase “thermal effect” refers to the biological effect of the ultrasonic energy delivered to the target tissue, typically within the conditions set forth in Table 2 hereinabove. Ultrasonic energy within those conditions will produce significant heat in the target site, typically raising the local temperature by from 10° C. to 40° C.

The phrases “mechanical index (MI)” and “thermal index (TI)” are defined as follows. The American Institute for Ultrasound in Medicine (AIUM) and the National Electrical Manufacturers Association (NEMA) in “Standard for Real-Time Display of Thermal and Mechanical Indices on Diagnostic Ultrasound Equipment”, 1991, have together defined the terms “mechanical index” MI and “thermal index” TI for medical diagnostic ultrasound systems operating in the frequency range of 1 to 10 MHz. Although therapeutic ultrasound is not included within the scope of this standard, the terms are useful in characterizing ultrasound exposure.

The mechanical index is defined as the peak rarefactional pressure P (in MPa) at the point of effectivity (corrected for attenuation along the beam path) in the tissue divided by the square root of the frequency F (in MHz), or

MI=P.[MPa]/sqrt (F [MHz])

The tolerated range for medical diagnostic equipment is up to an MI of 1.9. MI values above approximately unity represent acoustic levels which can cause mechanical bio-effects in human subjects.

The thermal index is defined as the average energy W (in mW) times the frequency F (in MHz), divided by the constant 210, or

TI=W[mW]*F[MHz]/210

A TI of 1 implies a temperature increase in normally vascularized muscle tissue of one Centigrade degree. The FDA standard for a maximum temperature of surface contact ultrasound devices is 41 degrees C. “Deep heat” ultrasound therapy devices may generate higher temperatures within tissue. In the vascular arena, however, even slight temperature excursions may cause unwanted formation and accumulation of clot. Moreover, increased temperature of tissue may cause inflammation in the area of treatment. Therefore, TI values in excess of four are generally considered the threshold for causing thermal bio-effects.

TI values as set forth herein may be calculated in accordance with the techniques described above. The TI parameter as defined represents a steady state condition, not a short term “transient” exposure. Using the assumption of 0.3 dB/MHz/cm as the energy absorption rate for normally vascularized muscle tissue, adequate doses of ultrasound can be delivered to achieve enhanced cellular absorption and/or transfection before thermal energy within the tissues generates an unacceptable temperature. Due to the difference in total energy absorption between the transient and continuous exposure, the AIUM definition of TI used herein refers to the continuous TI, as compared to the transient TI. While the calculated values of TI correspond to higher temperatures, the actual temperature increases will be lower because of the transient exposure employed by the methods of the present invention. Such shorter exposures even with relatively high values of TI will usually result in only modest temperature increases in tissue.

The methods of the present invention may be performed most simply by applying vibrational energy to tissue within the target site to which the nucleic acid vaccine has been introduced, under mechanical conditions, typically those conditions set forth in Table 1 hereinabove. It has been found that the application of such vibrational energy enhances the immune response of the human or other animal host even without any further exposure to other energy, such as thermal vibrational energy as set forth in Table 2. The mechanical vibrational energy may be applied for a time sufficient to enhance transfection, typically from 1 second to 3 minutes, usually from 20 seconds to 80 seconds, although the time of application may vary significantly. Exemplary devices and methods for applying mechanical vibrational energy which are useful in the present application are set forth in the co-pending application Ser. No. 09/435,095, the full disclosure of which has previously been incorporated herein by reference.

Usually, however, it will be preferable to combine the application of the mechanical vibrational energy with the application of thermal vibrational energy, typically under the conditions set forth in Table 2 hereinabove. The application of the thermal vibrational energy may be performed sequentially, simultaneously, or even before the injection and/or application of the mechanical vibrational energy. The thermal vibrational energy will be applied for a time sufficient to induce an injury immune response, typically for a time in the range from 1 second to 3 minutes, usually from 20 seconds to 80 seconds, although these time periods may vary widely and will depend on the level of applied power.

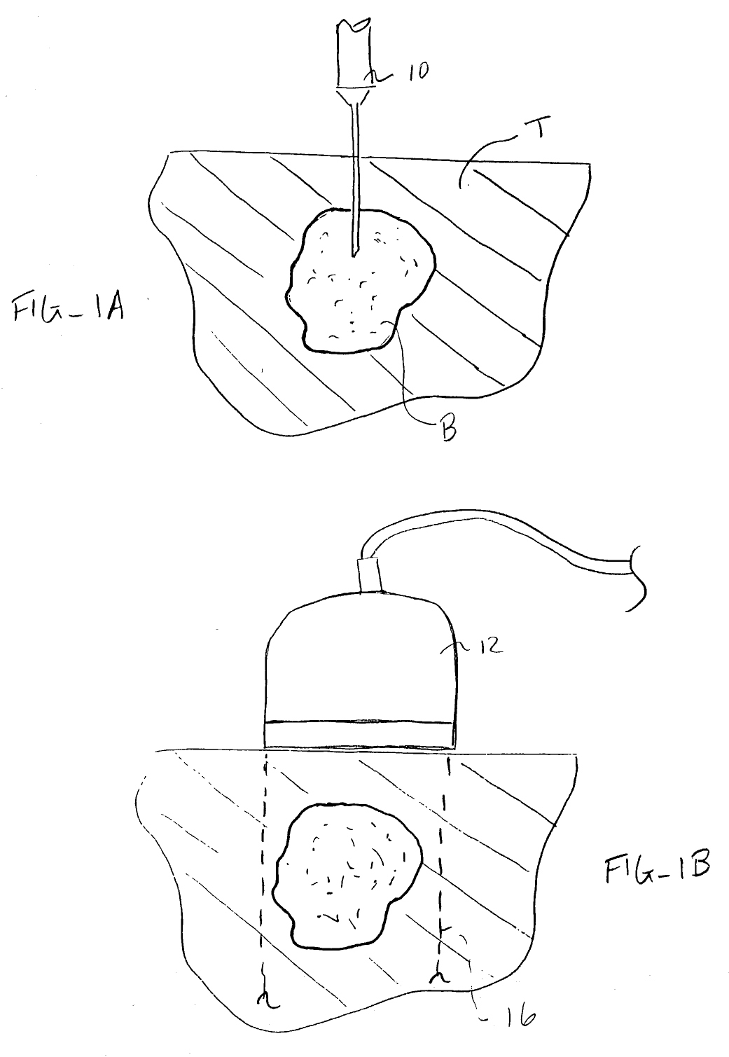

Referring now to FIGS. 1A-1C, a first exemplary protocol for performing a method according to the present invention is described. A needle and syringe 10, or other solid tissue injection device, is used to deliver a bolus B of a nucleic acid vaccine to solid tissue T of a patient to be immunized. After the nucleic acid vaccine has been injected, it will disperse in the tissue to occupy a volume (target site), typically within the ranges set forth above (FIG. 1A). Without further intervention, the nucleic acid vaccine would often transfect some of the cells within the target site, usually resulting in at least a low level of immune response. In some instances, however, there would be no detectable immune response.

According to the present invention, the immune response is enhanced by applying vibrational energy to the target site of the bolus B. As illustrated in FIG. 1B, the vibrational energy may be applied by a first transducer assembly 12 which applies vibrational energy under first conditions selected to enhance transfection of cells within the target site to produce antigen without significant tissue damage. Exemplary conditions have been set forth in Table 1 above. The enhanced transfection of cells will, by itself, improve the immune response by increasing the humoral and/or cellular responses. Thus, the present invention includes applying vibrational energy under the first conditions only, without necessarily applying vibrational energy under the second conditions.

It will be generally preferred, however, to also apply vibrational energy under the second conditions to the tissue in the target site. This may be done, for example, by engaging a second transducer assembly 14 (FIG. 1C) against the tissue surface above the target site incorporating the bolus B. Generally, the application of vibrational energy under the first conditions will be applied with a wide beam transducer 12 which produces a relatively wide, uniform ultrasonic or other vibrational field, as indicated by broken lines 16 in FIG. 1B. In contrast, the vibrational energy applied under the second conditions will usually be focused, applying a beam which narrows with increased intensity in the region of the target site of bolus B, as indicated by broken lines 18 in FIG. 1C.

As can be seen from FIGS. 1A-1C, the methods of the present invention may be carried out using separate instruments, such as the syringe and needle 10 (or other nucleic acid injection device), a first transducer assembly 12 which applies vibrational energy into the first conditions, and a second separate transducer assembly 14 which applies vibrational energy under the second conditions. The present invention will also comprise integrated systems which include any two or all three of these components, particularly where the injection device is combined with a source of the nucleic acid, e.g., the nucleic acid vaccine is provided in a separate vial to be loaded into the syringe or alternatively may be provided preloaded in the syringe or other injection device. Such systems may conveniently be packaged together in a common package, such as a box, tube, pouch, or the like, and will usually further be combined with written or electronic instructions for use (IFU) setting forth the methods for using the system to immunize a human or other animal host.

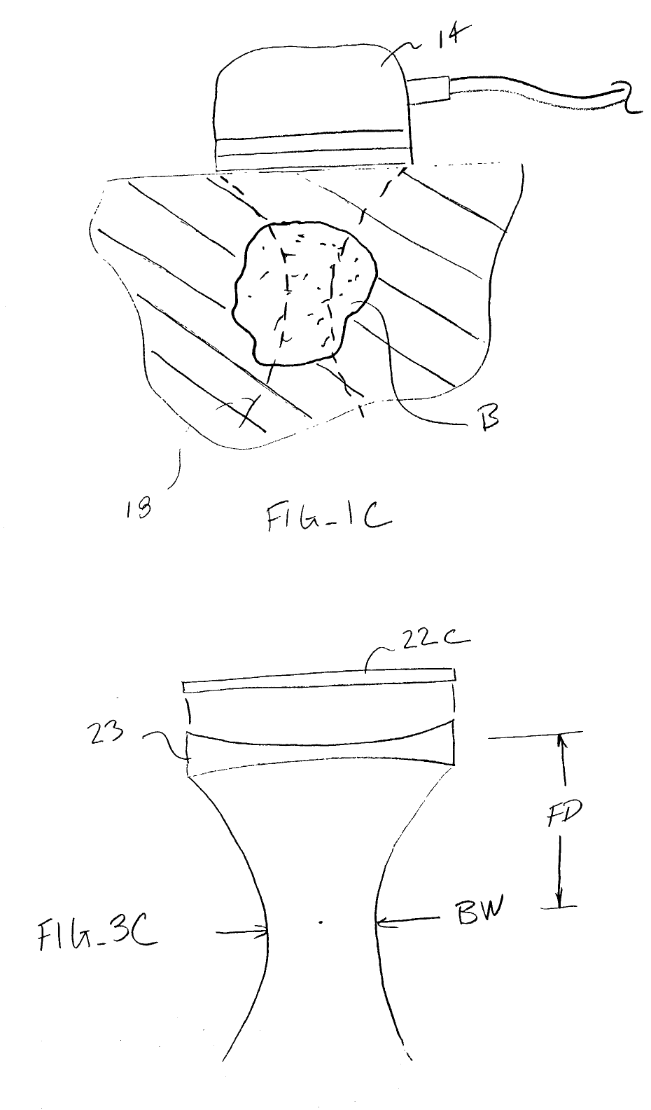

The first transducer assembly 12 may be constructed in accordance with the teachings of co-pending application Ser. No. 09/435,095, the full disclosure of which has been incorporated herein by reference. The construction of the second transducer assembly 14, however, will differ from that described in the co-pending application, since the desired vibrational conditions produced by the transducer will differ significantly. Referring now to FIG. 2, the second transducer assembly 14 may be constructed from a hand-held housing 20. The second transducer assembly 14, which is intended to produce thermal damage in the tissue in the target site, may best be implemented using a spherically concave transducer assembly 22 comprising a piezoelectric ceramic 24 and a matching layer 26. The transducer assembly 22 will be held in an annular mount 28 near the top of the housing 20 and will have an air backing region 30 overhead. The housing below the transducer assembly 22 will be filled with a coupling fluid 32, typically water, with an acoustically transparent window 34 positioned in an aperture at the lower end of the housing. The coupling fluid 32 may include agents to inhibit biological contamination, such as antibiotics, antimicrobial agents, antiseptics, and the like.

The radius R_{c} of the spherical transducer assembly 22 will produce a focal distance which is slightly short of the radius. Matching layer 26 will typically have a quarter wavelength thickness, being formed for example from loaded epoxy, in order to better couple acoustic (vibrational) energy from the piezoelectric ceramic or other transducer to the coupling fluid. The result will be a focused vibrational energy profile indicated by broken lines 36 with the thermal effect being concentrated within a target region TR surrounded by broken line 38. The precise geometry of the transducer assembly 14, of course, can be selected to provide the desired volume and tissue depth for the target region TR, usually corresponding to the depth of injection for the nucleic acid vaccine.

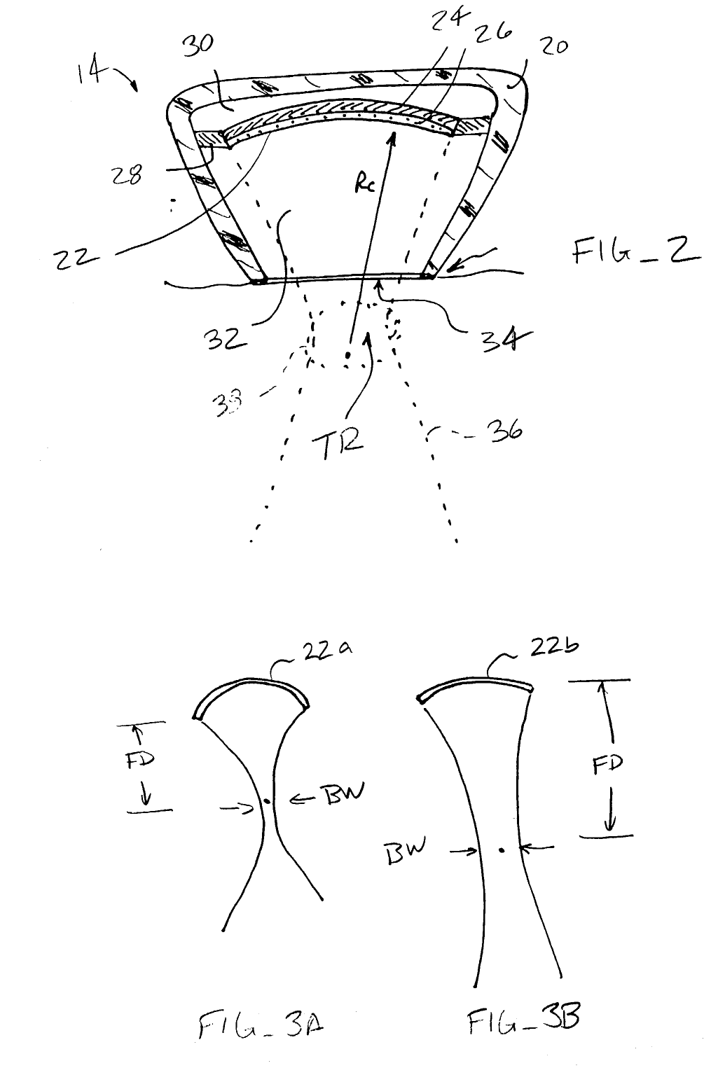

As illustrated in FIGS. 3A and 3B, the degree of concavity of the transducer assembly 22 may be selected to determine the focal distance FD and beam width BW. Transducer assemblies 22 having a smaller radius of curvature, such as shown in FIG. 3A, will have a shorter focal distance and narrower beam width. Transducer assemblies 22b, in contrast, having longer curvature radiuses will have longer focal distances FD and beam widths BW. A shorter focal length may be preferred to achieve desired thermal bioeffects in a smaller region of tissue, with less thermal damage to distal or lateral tissues. A longer focal length will result in thermal bioeffects to a larger tissue region, thereby generating a greater immune response.

Alternatively, focal characteristics of the second (or first) transducer may be achieved by the use of a planar transducer 22c and a focussing acoustic lens 23, as depicted in FIG. 3C. Acoustic lenses of a type suitable to focus ultrasound may be made from hard plastic materials, such as acrylics. Acoustic focussing lenses would typically be plano-concave or concave-concave (as illustrated). The use of acoustic lenses, however, is not as efficient as the use of spherically curved piezoelectrics due to acoustic reflection loses on both faces of the lenses and due to acoustic attenuation within the material itself.

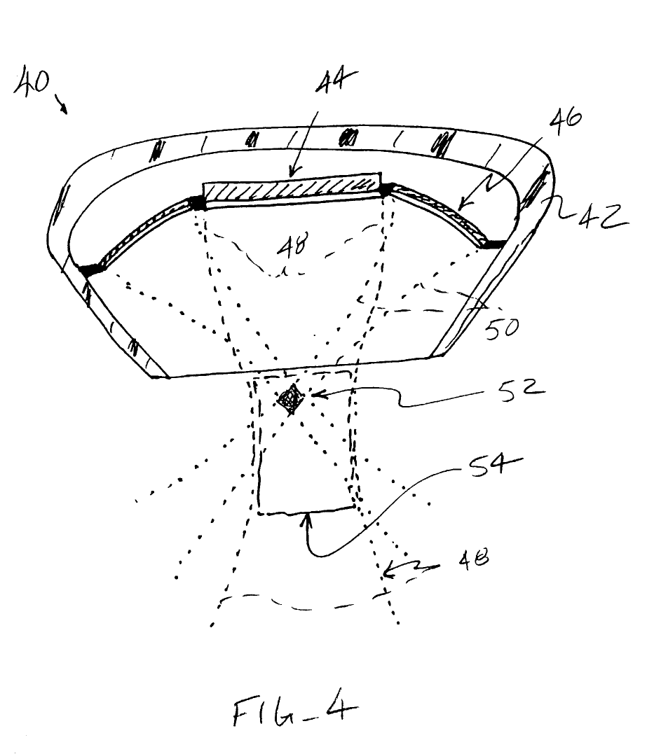

While it will be possible to perform the methods of the present invention using separate first and second transducer assemblies, as described above, it will often be preferred to provide transducer assemblies which are capable of producing vibrational energy under both the first conditions and the second conditions. Several designs for such combined transducer assemblies will now be described. Referring to FIG. 4, a transducer assembly 40 comprises a housing 42 having a central transducer 44 and an annular transducer 46. The general construction of the transducer assembly 40 will be similar to that of transducer assembly 14, incorporating the same air backing, coupling fluid, transparent window, and the like. The construction will differ in that the central transducer assembly 44 and annular transducer assembly 46 are configured to deliver mechanical vibrational energy and thermal vibrational energy, respectively. The central transducer assembly 44 will have a generally wide field of view, as indicated by broken lines 48 while the annular transducer 46 will have a more focused field of view which results in a target region 52 for the thermal vibrational energy. The mechanical energy, in contrast, will have a much larger target region 54, where the focused thermal region 52 is located within the larger mechanical region 54.

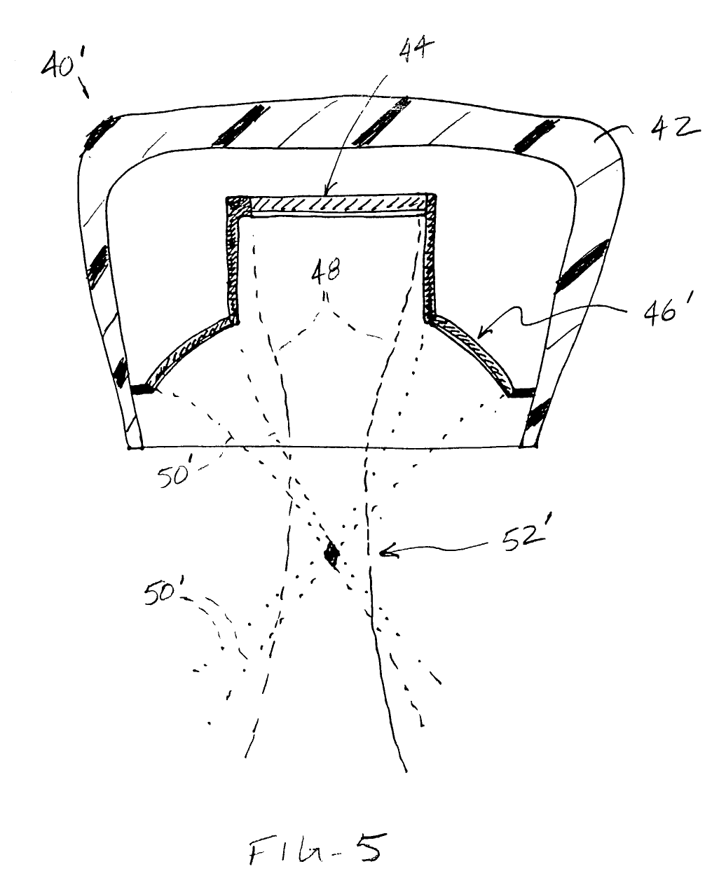

Referring now to FIG. 5, the depth of field of the thermal region 52′ may be increased by lowering the annular transducer 46′, as illustrated. Thus, the focus lines 50′ of the annular transducer 46′ will be lowered so that the thermal target region 52′ is itself lowered. In all other respects, construction of the transducer 40′ may be identical to that of the transducer 40 of FIG. 4. Such designs would be beneficial for injection at greater depths than the skin or organ surface. In some instances, it might be preferable to provide transducers having an adjustable depth of focus.

As a further modification of the combined mechanical/thermal devices of the present invention, a transducer assembly 70 having housing 72 may be constructed with a thermal transducer 74 mounted in the middle with an annular mechanical transducer 76. The thermal transducer 74 will preferably have a spherically concave construction so that it focuses the thermal vibrational energy as indicated by broken lines 78. The mechanical annular transducer 76 will similarly focus the mechanical vibrational energy along broken lines 80, so that the target regions of both the thermal and mechanical energy coincide at region 82. Such a construction would allow the thermal target region to have a longer depth of field while the beam width and depth of the mechanical field would be substantially reduced. Such a definition of the mechanical field could eliminate unwanted bioeffects of the vibrational transducers, particularly at the tissue-bone interface.

As yet a further variation of this concept of two ultrasound conditions, a first condition for the generation of mechanical bioeffects to enhance transfection rates and a second condition for the generation of a thermal bioeffect to enhance the immune response, it may be possible to utilize a single “hybrid” ultrasound condition, which may be less than optimal for transfection and immune response enhancements, but which achieves a sufficient net result to warrant implementation. As seen in Tables 1 and 2, the “general” conditions overlap (with the exception of TI), and potentially allow for a single device operating within the range overlaps to the extent possible to accomplish both ends.

TABLE 3
Combined Mechanical and Thermal Conditions

| Freq. | Intensity | Duty Cycle | | |
|---|---|---|---|---|
| (MHz) | (SPPA) | (%) | MI | TI |
| 1-5 | 10-2000 | 10-50 | 0.5-2 | 5-20 |

Thus it can be seen that a wide variety of combinations of mechanical and thermal transducer assemblies may be employed in the apparatus and methods of the present invention. Depending on the animal host and the nature of the target tissue, different depths and volumes of the target regions for both the mechanical vibrational energy and the thermal vibrational energy may be provided and combined in unique combinations.

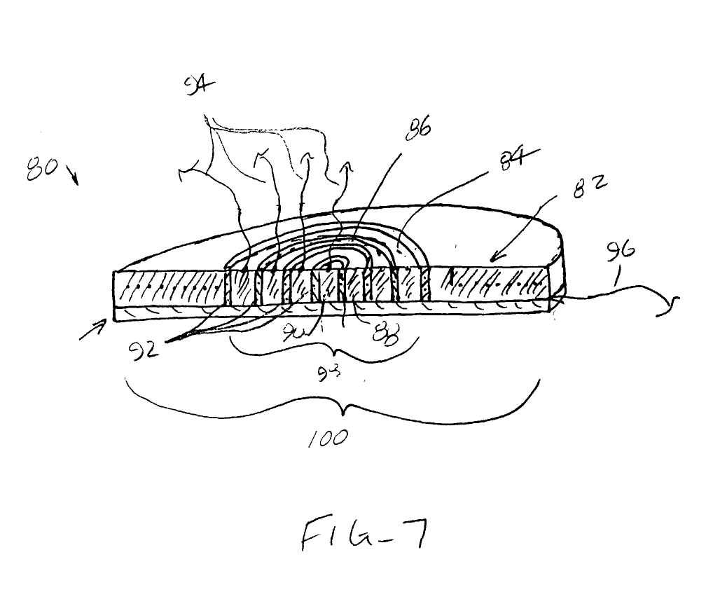

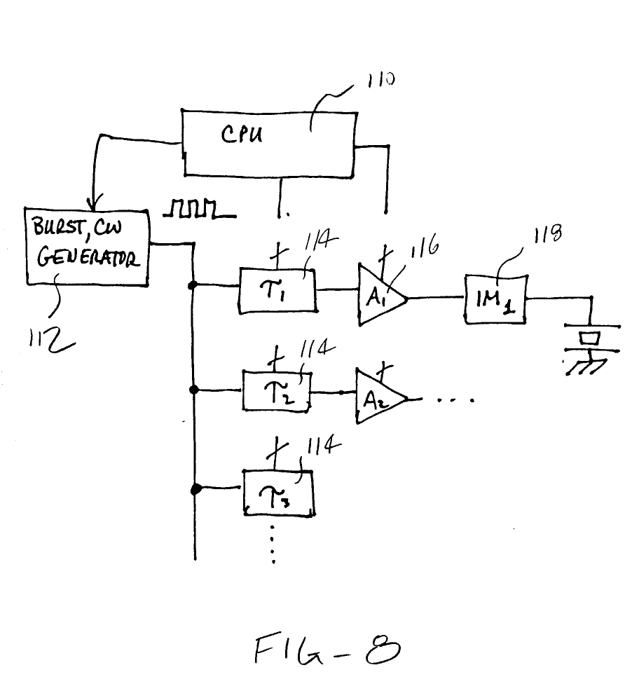

A wide variety of other transducer configurations could also be employed. For example, as illustrated in FIG. 7, a composite annular array intended to be driven at a single frequency is illustrated. The array 80 comprises a plurality of annular piezoelectric segments 82, 84, 86, 88, and 90, separated by isolation zones 92. Each of the piezoelectric segments 82-90 will be separately connected by a conductor 94, typically with a common ground layer connected by a conductor 96. Thus, each segment of the array can be energized by its own power amplifier and phase shifter, as illustrated in FIG. 8.

To achieve the mechanical vibrational effects according to the present invention, only the center elements 84-90 would be energized with a zero phase shift to produce a wide field of view 98. The elements 84-90 would be energized with a low duty cycle, typically 6%, and high amplitude. To achieve the thermal vibrational effects of the present invention, all elements 82-90 of the array would be energized, with phase shifts selected to achieve a narrow field of view and minimum depth of field. The elements would typically be driven in a continuous wave (CW) mode, but at a lower amplitude than was used for the mechanical effects.

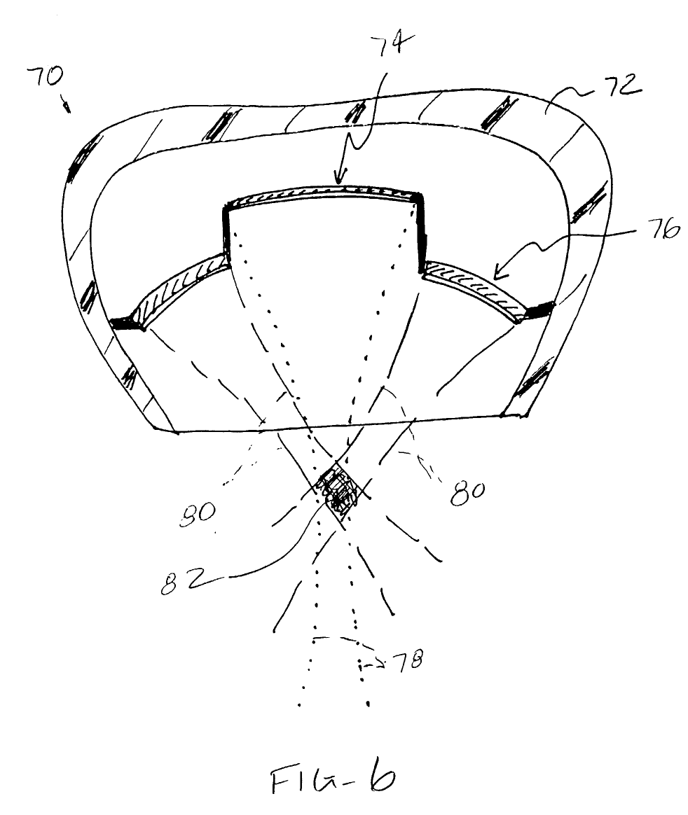

The power amplifier and phase shifting circuitry illustrated in FIG. 8 would include a microprocessor 110 to control the operation of the system components. A burst/CW generator 112 would selectively deliver either electronic bursts of a pre-selected frequency and burst repetition pattern (to achieve the mechanical vibrational effect) or would produce a continuous wave at the frequency selected to provide the thermal vibrational effect. The generator would include time delay circuits 114, amplifiers 116, and beams matching circuits 118 connected to each one of the annular segments 82-90 of the annular transducer array 80. Microprocessor 110 could selectively energize just the central transducer segments 84-90 for operation in the thermal mode, providing particular time delays and gains on the amplifiers to generate a Fresnel aperture. Alternatively, the amplifiers might be set to full power with the time delay set to approximate a spherically concave radiative surface. The time delays need only delay by increments of 1/16 of a wavelength for adequate beam formation. Since variable time delays might be used only in the CW mode of operation, the maximum duration of the time delays need be no longer than 1 wavelength. Alternatively, the annular array may contain elements, e.g. piezoelectric sections, which operate at different frequencies which can be arranged to emulate the devices of FIGS. 4-6. Beam characteristics can thus be modified for the treatment of individual patients, for optimal mechanical and thermal effects in the injected organ, and the like. Additionally, array timing may be adjusted during the course of therapy to dynamically sweep the beam through tissues. As a further alternative, the transducer may comprise a two-dimensional phased array with individual elements operating at the same or at different frequencies. Actuation of the elements can be timed by a programmable controller to achieve specific mechanical and thermal beam profiles.

In all of the above embodiments, it will be appreciated that the transducer assemblies may be provided with central passages in order to permit introduction of an injection needle or other nucleic acid vaccine administering device. As an additional alternative, the apparatus of the present invention may be integrally combined with needles or other administering devices, generally as taught in co-pending application Ser. No. 09/435,095, the full disclosure of which has previously been incorporated herein by reference.

What is claimed is:

1. A method for enhancing an immune response in an animal, said method comprising:
introducing nucleic acids encoding one or more immunogens to a target site in tissue; and
applying vibrational energy to the target site under at least two different conditions selected to enhance transfection or to produce an inflammatory response.

2. The method of claim 1, wherein applying comprises applying to at least a portion of the target site vibrational energy under first conditions selected to enhance transfection of cells to produce antigen without significant tissue damage.

3. The method of claim 2, wherein applying further comprises applying to at least a portion of the target site vibrational energy under second conditions selected to produce tissue damage to stimulate an inflammatory response to the antigen produced by the transfected cells.

4. The method of claims 1, 2, or 3, wherein said animal is a human.

5. The method of claims 1, 2, or 3, wherein said nucleic acids encode an immunogen which is a protein or peptide of a pathogen.

6. The method of claim 5, wherein said pathogen is selected from the group consisting of a bacterium, a fungus, a yeast, a protozoan, and a virus.

7. The method of claim 6, wherein said pathogen is a bacterium selected from the group consisting of an enteric bacterium, Clostridium, Vibrio, Nocardia, Corynebacterium, Listeria, Legionella, Bacilli, Staphylococcus, Streptococci, Borrelia, Mycobacterium, Neisserium and Trepanema bacterium.

8. The method of claim 6, wherein said pathogen is a fungus selected from the group consisting of Dermatophyte, Pneumocystis, Trypanosoma, Plasmodium, Candida, Cryptococcus, Histoplasma, Coccidioide, an Amoeba and Schistosome.

9. The method of claim 6, wherein said pathogen is a virus selected from the group consisting of parvovirus, an orthomyxovirus, paramyxovirus, and picomavirus, papovirus, herpesvirus, togavirus, and retrovirus.

10. The method of claim 9, wherein said pathogen is the retrovirus HIV.

11. The method of claim 10, wherein the nucleic acid vaccine encodes one or more HIV proteins or peptides.

12. The method of claim 11, wherein said HIV protein or peptide is the HIV gag protein or a peptide fragment thereof.

13. The method of claim 11, wherein said nucleic acid introduced encodes both (a) an HIV gag protein or a peptide fragment thereof and (b) an HIV env protein or a peptide fragment thereof.

14. The method of claim 13, wherein said nucleic acid introduced comprises a codon-optimized gag-encoding region and a codon-optimized env-encoding region.

15. The method of claims 1, 2, or 3, wherein said nucleic acid vaccine encoding one or more immunogens of interest is administered to said animal incorporated in a plasmid form.

16. The method of claims 1, 2, or 3, wherein said nucleic acid vaccine encoding one or more immunogens of interest is administered to said animal associated with protein or lipid.

17. The method of claims 1, 2, or 3, wherein said nucleic acid vaccine is introduced to said animal by intramuscular or intradermal injection.

18. The method of claim 3, wherein the first conditions comprise a target site field having a volume in the range from 0.1 cm³ to 5 cm³, a thermal index selected to raise the temperature in the target site by from 10° C. to 40° C., and a mechanical index from 0.5 to 20.

19. The method of claim 18, wherein the second conditions comprise a target site field having a volume in the range from 0.01 cm³ to 1 cm³, a thermal index selected to raise the temperature in the target site by from 10° C. to 40° C., and a mechanical index in the range from 0.1 to 2.

20. The method of claim 19, wherein the first conditions comprise a field intensity which varies in intensity by less than 6 db in a lateral direction across the width of the field and across the depth of the target site.

21. A system for enhancing an immune response in an animal, said system comprising:
means for administering a nucleic acid vaccine to a target site in tissue in the animal;
means for applying vibrational energy to the target site; wherein the vibrational energy applying means produces energy which both enhances transfection and which induces an inflammatory response, and
a source of nucleic acid vaccine coupleable to the administering means.

22. A system as in claim 21, wherein the vibrational energy applying means comprises a first transducer which directs vibrational energy at the target site under conditions which enhance transfection of cells in the target site with nucleic acids delivered to the target site by the administering means to produce an antigen encoded by the nucleic acid vaccine.

23. Apparatus as in claim 21, wherein the vibrational energy applying means further comprises a second transducer which directs vibrational energy at the target site under conditions which produce tissue damage to stimulate an immune response to the antigen produced by the transfected cells.

24. A system as in claim 23, further comprising a housing, wherein the first and second transducers are disposed in the housing.

25. A system as in claims 22, 23, or 24, wherein the first transducer operates at a thermal index selected to raise the temperature in the target site by from 10° C. to 40° C., and a mechanical index in the range from 0.5 to 20.

26. A system as in claim 25, wherein the second transducer operates at a thermal index selected to raise the temperature in the target site by from 10° C. to 40° C., and a mechanical index in the range from 0.1 to 2.

27. A system as in claim 24, wherein the second transducer is arranged annularly about the first transducer and focuses vibrational energy at a region within a beam produced by the first transducer.

28. A system as in claims 21, 22, 23, or 24, wherein the nucleic acid vaccine encodes an immunogen which is a protein or peptide of a pathogen.

29. A system as in claim 28, wherein said pathogen is selected from the group consisting of a bacterium, a fungus, a yeast, a protozoan, and a virus.

30. A system as in claim 29, wherein said pathogen is a bacterium selected from the group consisting of an enteric bacterium, a Clostridium, a Vibrio, a Nocardia, a Corynebacterium, a Listeria, a Legionella, a Bacilli, a Staphylococcus, a Streptococci, a Borrelia, a Mycobacterium, a Neisserium and a Trepanema bacterium.

31. A system as in claim 29, wherein said pathogen is a fungus selected from the group consisting of a Dermatophyte, a Pneumocystis, a Trypanosoma, a Plasmodium, a Candida, a Cryptococcus, a Histoplasma, a Coccidioide, an Amoeba and a Schistosome.

32. A system as in claim 29, wherein said pathogen is a virus selected from the group consisting of a parvovirus, an orthomyxovirus, a paramyxovirus, and picornavirus, a papovirus, a herpesvirus, a togavirus, and a retrovirus.

33. A system as in claim 32, wherein said pathogen is the retrovirus HIV.

34. A system as in claim 33, wherein the DNA administered in step (a) encodes one or more HIV proteins or peptides.

35. A system as in claim 34, wherein said HIV protein or peptide is the HIV gag protein or a peptide fragment thereof.

36. A system as in claim 21, wherein the nucleic acid vaccine encoding said one or more immunogens of interest is incorporated in a plasmid form.

37. A system as in claim 21, wherein the nucleic acid vaccine encoding one or more immunogens of interest is associated with protein or lipid.

38. A system as in claim 21, wherein said means for administering the nucleic acid vaccine to said animal accomplishes intramuscular or intradermal administration of said DNA.

39. A system as in claim 23, wherein said means for administering said DNA is a device selected from the group consisting of a needle and a nucleic acid gun.

40. A system as in claim 39, wherein the applying means comprises a first hand held device which incorporates the first transducer and a second handheld device, separate from the first device, incorporating the second transducer.

41. A system as in claim 39, wherein the applying means comprise a single hand held device incorporating both the first and second transducers, wherein said first and second transducers are arranged such that vibrational energy from the first transducer spans a large volume while vibrational energy from the second transducer is focused within the large volume.

42. A method for enhancing an immune response in an animal, said method comprising:
introducing nucleic acids encoding one or more immunogens to a target site in tissue; and
applying vibrational energy to the target site under at least one set of conditions selected to enhance transfection and to produce an inflammatory response.

43. A system for enhancing an immune response in an animal, said system comprising:
means for administering a nucleic acid vaccine to a target site in tissue in the animal;
means for applying vibrational energy to the target site, wherein the vibrational energy applying means produces energy under a single set of conditions, which both enhances transfection and which induces an inflammatory response; and
a source of nucleic acid vaccine coupleable to the administering means.
